(19) United States
(12) Patent Application Publication
(10) Pub. No.: US 2018/0110890 A1
(43) Pub. Date: Apr. 26, 2018

(54) ULTRAVIOLET LIGHT STERILIZER

(71) Applicant: TOKUYAMA CORPORATION, Shunan-shi, Yamaguchi (JP)

(72) Inventor: Shingo MATSUI, Shunan-shi (JP)

(73) Assignee: TOKUYAMA CORPORATION, Shunan-shi, Yamaguchi (JP)

(21) Appl. No.: 15/561,838

(22) PCT Filed: Mar. 18, 2016
(86) PCT No.: PCT/JP2016/058818

§ 371 Date: Sep. 26, 2017

(30) Foreign Application Priority Data
Mar. 31, 2015 (JP) 2015-074477

(51) Int. Cl. A61L 2/10; G01J 1/02

(52) U.S. Cl. CPC A61L 2/10; A61L 2209/12; A61L 2202/14; G01J 1/0252

(57) ABSTRACT

An ultraviolet light sterilizer includes: a deep ultraviolet light source comprising one or more deep ultraviolet light emitting diode and irradiating a deep ultraviolet ray toward a work; and a controller controlling (1) an irradiation time t (unit: second) defined as a time for which the deep ultraviolet ray is irradiated to the work, (2) a distance d (unit: cm) between the work and the deep ultraviolet light source, or (3) an emission power P (unit: mW) of the deep ultraviolet light source, or combination thereof, such that an integral irradiance I (unit: mJ/cm²) of the deep ultraviolet ray irradiated to the work during the irradiation time reaches a predetermined value I₀.

### TECHNICAL FIELD

The present invention relates to sterilizers using ultraviolet rays.

### BACKGROUND ART

Ultraviolet light sterilization causes no residues differently from chemical sterilization, satisfies a high level of safety, and seldom brings about changes in irradiated objects. Thus, it is proposed to use ultraviolet light sterilization for sterilizing objects in various scenes.

For example, Patent Literature 1 describes a conveyer type sterilizer using a xenon flash lamp, wherein a UV irradiation room part is arranged in the middle of a conveyer surface that carries an object to be sterilized which is to be irradiated with UV, an upstream shielding duct part and a downstream shielding duct part with the conveyor surface as their bottom faces are continuously arranged at an inlet and an outlet of the UV irradiation room part, and a shielding plate to pass the object to be sterilized without making the object contact with the inside is arranged in each shielding duct part.

However, discharge tubes such as xenon flash lamps and low-pressure mercury lamps have short lives although they are able to irradiate ultraviolet rays of a high intensity. Thus, discharge tubes have to be replaced periodically, which invites increased running costs.

In recent years, light emitting diodes that emit ultraviolet light (ultraviolet light emitting diodes) have been developed as ultraviolet light sources instead of discharge tubes, and it has been proposed to apply ultraviolet light emitting diodes for sterilization use. For example, Patent Literature 2 discloses an optical sterilization method by flashing light pulses of emitting the flashing light pulses to a target to be sterilized, the method comprising driving a blue light emitting diode array by predetermined pulse signals, letting the flashing light pulses emit from the blue light emitting diode array, and irradiating a near ultraviolet ray to the target. It is considered that according to such an ultraviolet light sterilizer having an ultraviolet light emitting diode as an ultraviolet light source, running costs can be reduced since blue and other ultraviolet light emitting diodes have much longer lives than conventional discharge tubes.

### CITATION LIST

### Patent Literature

Patent Literature 1: JP 2005-312978A

Patent Literature 2: JP 2004-275335A

Patent Literature 3: JP 2005-93566A

Patent Literature 4: JP 2007-97251A

Patent Literature 5: JP 2010-4691A

Patent Literature 6: JP 5591305B

### SUMMARY OF INVENTION

### Technical Problem

However, in the flashing light sterilization using a blue light emitting diode disclosed in Patent Literature 2, the obtained sterilization effect of 2-hour flashing light irradiation is only 40%.

In recent years, development of deep ultraviolet light emitting diodes that emit ultraviolet light of a much shorter wavelength than conventional blue and ultraviolet light emitting diodes is in progress, and one may think of performing sterilization using deep ultraviolet light emitting diodes. However, deep ultraviolet light emitting diodes are lower than conventional blue or ultraviolet light emitting diodes in emission intensity. Thus, microorganisms on the target might not completely die out even if subjected to light irradiation for a long time.

An object of the present invention is to provide an ultraviolet light sterilizer using a deep ultraviolet light emitting diode as an ultraviolet light source, which enables sure sterilization. An ultraviolet surface light source that can be preferably employed in the ultraviolet light sterilizer is also provided.

### Solution to Problem

A first aspect of the present invention is an ultraviolet light sterilizer comprising:

a deep ultraviolet light source comprising one or more deep ultraviolet light emitting diode and irradiating a deep ultraviolet ray toward a work; and

a controller controlling (1) an irradiation time t (unit: second) defined as a time for which the deep ultraviolet ray is irradiated to the work, (2) a distance d (unit: cm) between the work and the deep ultraviolet light source, or (3) an emission power P (unit: mW) of the deep ultraviolet light source, or combination thereof, such that an integral irradiance I (unit: mJ/cm²) of the deep ultraviolet ray irradiated to the work during the irradiation time reaches a predetermined value I₀.

In the first aspect of the present invention, the emission power P can be controlled by controlling a forward current of the deep ultraviolet light emitting diode. For example, the forward current is preferably controlled using a boost DC-DC converter or a charge pump that can let a forward current of 10 mA to 1000 mA, preferably 50 mA to 500 mA flow with a battery of approximately 1.5 to 15V in voltage.

In one embodiment of the first aspect of the present invention, the ultraviolet light sterilize further comprises: a temperature sensor measuring a device temperature of the deep ultraviolet light emitting diode, wherein the controller controls the emission power P based on the device temperature of the deep ultraviolet light emitting diode measured by the temperature sensor.

In one embodiment of the first aspect of the present invention, the controller controls the distance d and/or the emission power P, such that the integral irradiance I reaches the predetermined value I₀.

In one embodiment of the first aspect of the present invention, the ultraviolet light sterilizer further comprises: a distance sensor measuring the distance d.

In this embodiment, preferably, the controller controls the irradiation time t and/or the emission power P based on the distance d measured by the distance sensor.

In one embodiment of the first aspect of the present invention, the predetermined value I₀ is no less than 50.0 mJ/cm². This value is enough for common microorganisms to die out.

In one embodiment of the first aspect of the present invention, the predetermined value I₀ is determined based on a microorganism to be killed.

In this embodiment, preferably, the ultraviolet light sterilizer further comprises: an input device adapted for determining the microorganism to be killed, and preferably, the predetermined value I₀ is a value such that no less than 99% of the microorganism dies out.

In one embodiment of the first aspect of the present invention, the deep ultraviolet light source comprises: a substrate; and a plurality of the deep ultraviolet light emitting diodes arranged on the substrate.

In one embodiment of the first aspect of the present invention, the ultraviolet light sterilizer further comprises: a housing; a support arranged in the housing, the support being adapted for placing the work on the support, wherein the deep ultraviolet light source is arranged in the housing and is arranged opposite to the support; and a driver rotating the deep ultraviolet light source relative to the support such that a dose of the deep ultraviolet ray irradiated to the work is leveled.

In one embodiment of the first aspect of the present invention, the ultraviolet light sterilizer further comprises: a housing; a support arranged in the housing, the support being adapted for placing the work on the support, wherein the deep ultraviolet light source is arranged in the housing and is arranged opposite to the support; and a driver rotating the support relative to the deep ultraviolet light source such that a dose of the deep ultraviolet ray irradiated to the work is leveled

In one embodiment of the first aspect of the present invention, the deep ultraviolet light source comprises: a light guide plate; and a plurality of the deep ultraviolet light emitting diodes arranged on an end of the light guide plate.

A second aspect of the present invention is a deep ultraviolet surface light source comprising: a light guide plate; deep ultraviolet light emitting diodes arranged on an end of the light guide plate; a temperature sensor or a temperature controller, the temperature sensor measuring a device temperature of the deep ultraviolet light emitting diodes, and the temperature controller controlling the device temperature of the deep ultraviolet light emitting diodes; and a current controller controlling a forward current of the deep ultraviolet light emitting diodes, based on the device temperature of the deep ultraviolet light emitting diodes, such that an irradiance of a deep ultraviolet ray emitted from the light guide plate at a predetermined distance from the light guide plate reaches a predetermined value.

A third aspect of the present invention is a method for ultraviolet light sterilization of a work by means of a deep ultraviolet light source,

the deep ultraviolet light source comprising one or more deep ultraviolet light emitting diode and emitting a deep ultraviolet ray toward the work,

the method comprising:

irradiating a deep ultraviolet ray to the work from the deep ultraviolet light source for an irradiation time t at an emission power P, while controlling (1) the irradiation time t (unit: second) defined as a time for which the deep ultraviolet ray is irradiated to the work, (2) a distance d (unit: cm) between the work and the deep ultraviolet light source, or (3) the emission power P (unit: mW) of the deep ultraviolet light source, or combination thereof, such that an integral irradiance I (unit: mJ/cm²) of the deep ultraviolet ray irradiated to the work during the irradiation time reaches a predetermined value I₀.

In one embodiment of the third aspect of the present invention, the method for ultraviolet light sterilization may comprise: determining the integral irradiance I₀ (unit: mJ/cm²) of the deep ultraviolet ray to be irradiated to the work; setting the irradiation time t; calculating the emission power P necessary for the integral irradiance I of the deep ultraviolet ray irradiated to the work during the irradiation time t to reach the integral irradiance I₀; adjusting the emission power P and/or the distance d; and irradiating the deep ultraviolet ray from the deep ultraviolet light source to the work at the emission power P for the irradiation time t.

In one embodiment of the third aspect of the present invention, the method for ultraviolet light sterilization may comprise: determining the integral irradiance I₀ (unit: mJ/cm²) of the deep ultraviolet ray to be irradiated to the work; calculating the irradiation time t necessary for the integral irradiance I to reach the integral irradiance I₀ given that the deep ultraviolet ray is irradiated from the deep ultraviolet light source at the emission power P, based on the emission power P and the distance d; and irradiating the deep ultraviolet ray from the deep ultraviolet light source to the work at the emission power P for the irradiation time t.

In one embodiment of the third aspect of the present invention, the deep ultraviolet light source being arranged opposite to a conveyor, the work being placed on the conveyor, the method may comprise: starting to drive the conveyor, wherein the work is placed on the conveyor; measuring the distance d; calculating the irradiation time t necessary for the integral irradiance I to reach the predetermined value I₀ given that the deep ultraviolet ray is irradiated from the deep ultraviolet light source at the emission power P, based on the emission power P and the measured distance d; calculating a velocity to move the work while irradiating the deep ultraviolet ray to the work, based on the irradiation time t; and irradiating the deep ultraviolet ray from the deep ultraviolet light source to the work at the emission power P, while driving the conveyor at the calculated velocity.

In one embodiment of the third aspect of the present invention, the deep ultraviolet light source being arranged opposite to a conveyor, the work being placed on the conveyor, the method may comprise: driving the conveyor at a predetermined velocity, wherein the work is placed on the conveyor; measuring the distance d; calculating the emission power P necessary for the integral irradiance I to reach the predetermined value I₀, wherein the integral irradiance I is an integral irradiance of the deep ultraviolet ray irradiated to the work while the work passes through a region opposite to the deep ultraviolet light source; and irradiating the deep ultraviolet ray to the work from the deep ultraviolet light source at the emission power P, while driving the conveyor at the predetermined velocity.

### Advantageous Effects of Invention

According to the first aspect of the present invention, an ultraviolet light sterilizer using a deep ultraviolet light emitting diode as an ultraviolet light source, which enables sure sterilization can be provided.

The ultraviolet surface light source according to the second aspect of the present invention can be preferably used as the deep ultraviolet light source in the ultraviolet light sterilizer according to the first aspect of the present invention.

### BRIEF DESCRIPTION OF DRAWINGS

FIG. 1 is an explanatory schematic perspective view of an ultraviolet light sterilizer 100 according to one embodiment of the present invention, where a housing 10 is transparent.

FIG. 2 is a front cross-sectional view of the ultraviolet light sterilizer 100.

FIG. 3 is a view taken along the line A-A in FIG. 2.

FIG. 4 is a view taken along the line B-B in FIG. 2.

FIG. 5 is an explanatory flowchart of a control process S1 of the ultraviolet light sterilizer 100.

FIG. 6 is an explanatory schematic cross-sectional view of an ultraviolet light sterilizer 200 according to another embodiment of the present invention.

FIG. 7A is a plan view of a deep ultraviolet surface light source 220, and FIG. 7B is a side view of the deep ultraviolet surface light source 220.

FIG. 8 is an explanatory flowchart of a control process S2 of the ultraviolet light sterilizer 200.

### DESCRIPTION OF EMBODIMENTS

The above described effects and advantages of the present invention are made clear by the description of embodiments described below. Hereinafter the embodiments of the present invention will be described with reference to drawings. It is noted that the present invention is not limited to these embodiments. It is also noted that in the drawings, some reference numerals are omitted.

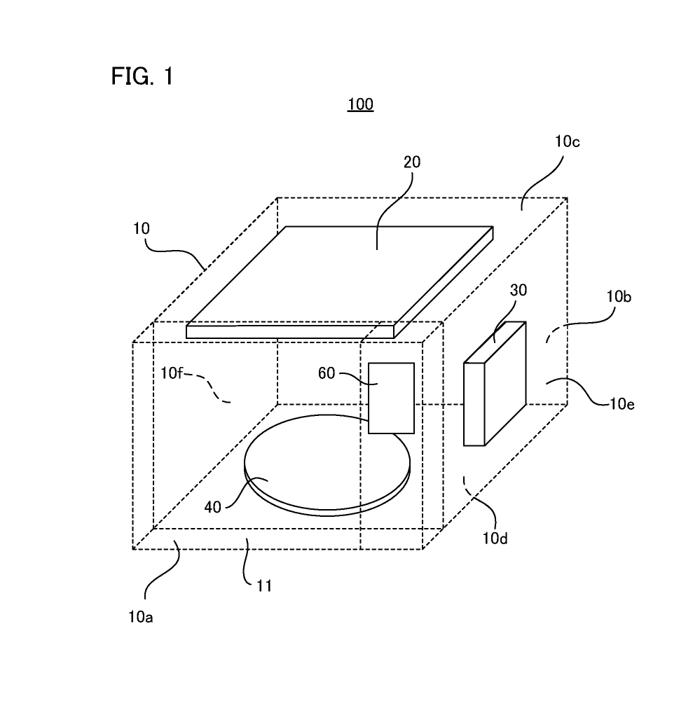

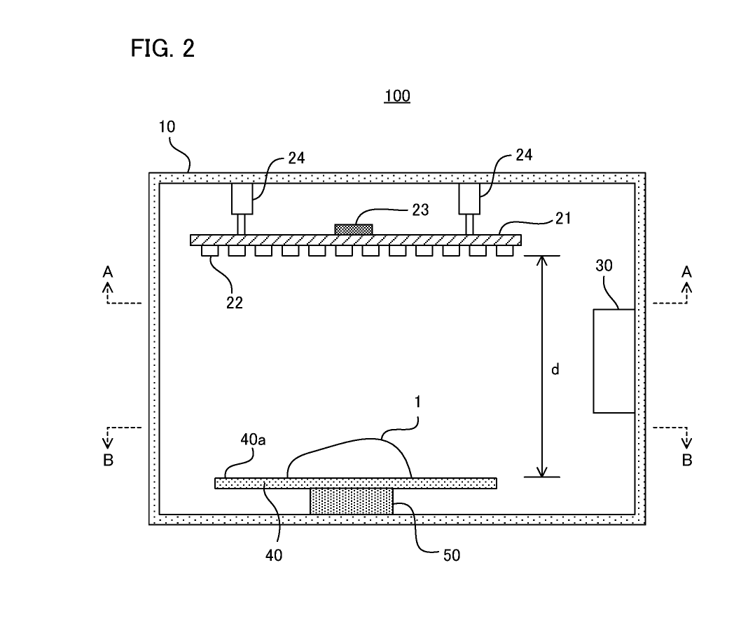

FIG. 1 is an explanatory schematic perspective view of an ultraviolet light sterilizer 100 according to one embodiment of the present invention, where a housing 10 (which will be described below) is transparent. FIG. 2 is a front cross-sectional view of the ultraviolet light sterilizer 100. The ultraviolet light sterilizer 100 includes a box-shaped housing 10 having a front face 10a, a back face 10b, a top face 10c, a bottom face 10d, and side faces 10e and 10f; a deep ultraviolet light source 20 arranged in the vicinity of the top face 10c inside the housing 10; a controller 30 arranged inside the housing 10; a support 40 for placing a work 1 thereon, which is arranged in the vicinity of the bottom face 10d inside the housing 10; a driver 50 arranged in the bottom face 10d side of the support 40, to rotate the support 40; and an input/output device 60 arranged outside the housing 10 (on the front face 10a in FIG. 1). An openable and closable door 11 is provided for the front side 10a of the housing 10. The work 1 can be put in and taken out of the housing 10 by opening of the door 11.

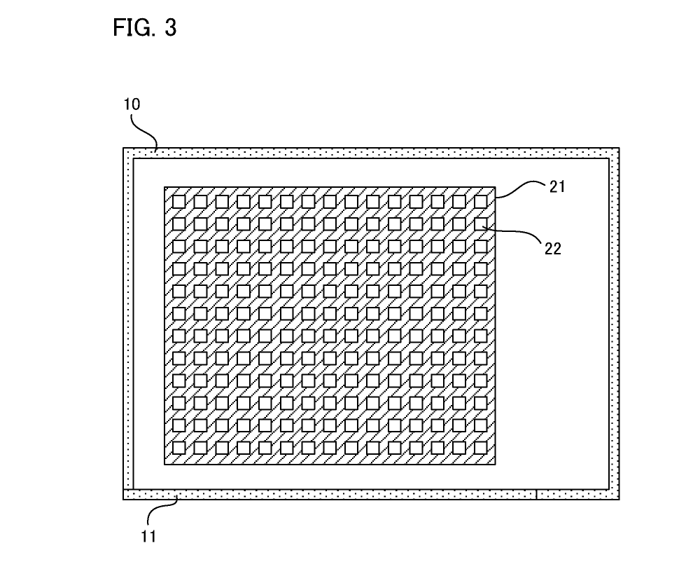

As shown in FIG. 2, the deep ultraviolet light source 20 is arranged in the housing 10 and opposite to a supporting surface 40a of the support 40. FIG. 3 is a view taken along the line A-A in FIG. 2. As shown in FIG. 3, the deep ultraviolet light source 20 has a substrate 21, a plurality of deep ultraviolet light emitting diodes 22, 22, . . . arranged on the substrate 21 (hereinafter may be simply referred to as “deep ultraviolet light emitting diodes 22”), and a temperature sensor 23 (for example, a thermoelectric thermometer etc.) that is provided so that device temperature of the deep ultraviolet light emitting diodes 22, 22, . . . can be measured. The deep ultraviolet light emitting diodes 22 are light emitting diodes each having an emission wavelength of 200 to 300 nm, preferably 220 to 270 nm. The deep ultraviolet light source 20 is fixed to the top face 10c of the housing 10 via actuators 24. The distance d between the work 1 and the deep ultraviolet light source 20 is adjusted by operation of the actuators 24. As shown in FIG. 2, in the ultraviolet light sterilizer 100, the distance between the ultraviolet light source 20 and the supporting surface 40a of the support 40 (that is, the maximum value of the distance between the surface of the work 1 and the deep ultraviolet light source 20) is employed as the distance d.

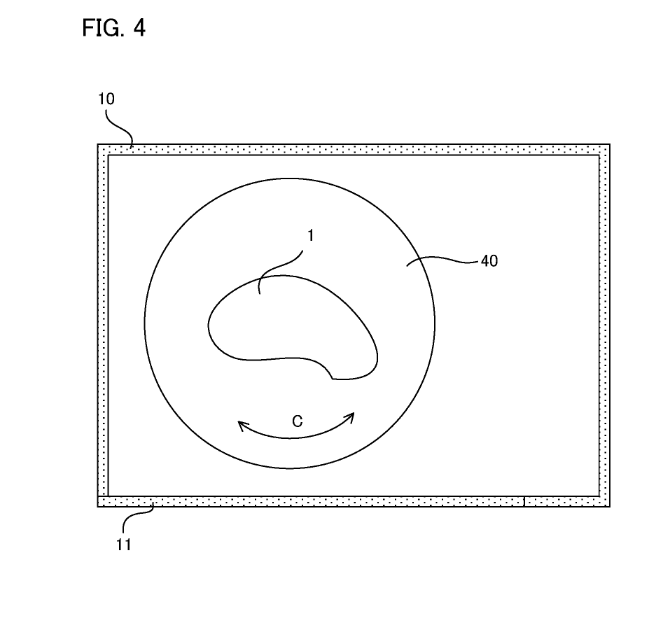

FIG. 4 is a view taken along the line B-B in FIG. 2, and shows the support 40. The support 40 is rotated by the driver 50 as shown by the arrow C in FIG. 4. Whereby, the dose of deep ultraviolet rays irradiated to the work 1 that is placed on the support 40 is leveled.

The input/output device 60 is configured so that information input by an operator can be transferred to the controller 30, and information transferred by the controller 30 can be displayed for an operator. At least, the input/output device 60 can receive information on a predetermined value I₀ and transfer the received information to the controller 30, and can receive input of a command for starting a sterilization process and transfer the received information on the input of the command to the controller 30. The input/output device 60 can also display information on completion of a sterilization process, which is transferred from the controller 30, for an operator. Examples of the input/output device 60 include any embodiment having a display such as a liquid crystal display, and an input device such as a keyboard.

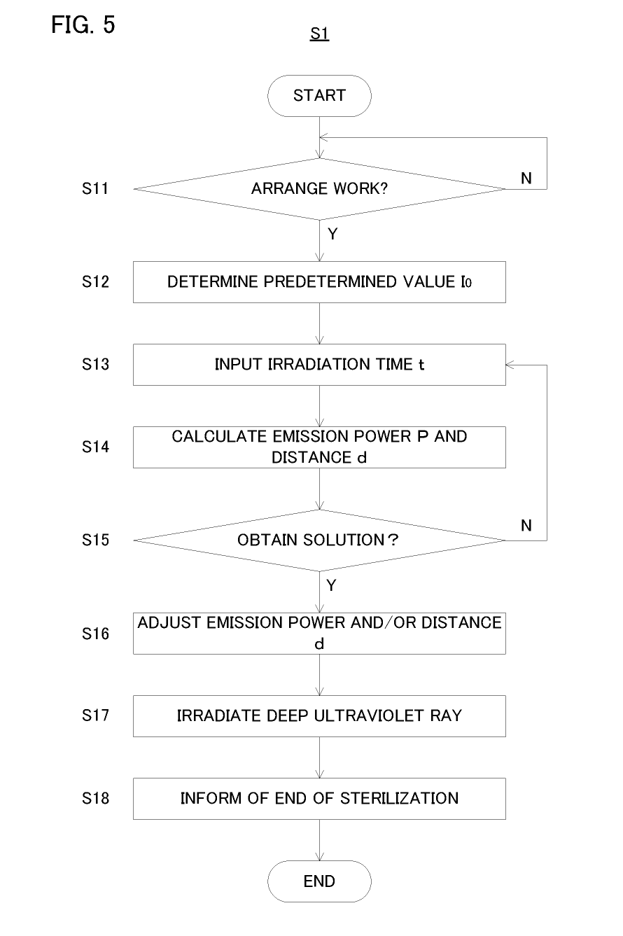

The controller 30 is connected to a driving circuit for the deep ultraviolet light emitting diodes 22, the temperature sensor 23, the actuators 24, the driver 50, and the input/output device 60, to control their operations. FIG. 5 is an explanatory flowchart of a control process S1 of the ultraviolet light sterilizer 100. FIG. 5 is also an explanatory flowchart of the method for ultraviolet light sterilization according to one embodiment of the present invention at the same time. The control process S1 includes steps S11 to S18. Operation of the controller 30 will be described with reference to FIG. 5.

In step S11, it is determined whether the work 1 is arranged on the supporting surface 40a of the support 40 or not. For an example, a gravimetric sensor etc. arranged on the bottom face of the support 40 can be used for this determination. For another example, an operator himself/herself may input information that the operator has arranged the work 1 on the support 40, to the controller 30 via the input/output device 60. If an affirmative judgment is made in step S11, the process moves to next step S12.

In step S12, the integral irradiance I₀ (unit: mJ/cm²) of deep ultraviolet rays to be irradiated to the work 1 is determined. In step S12, the controller 30 prompts an operator to select either a specific microorganism to die out, or a general sterilization process, via the input/output device 60. If a specific microorganism to die out is selected, the controller 30 assigns a dose with which no less than 99% of the microorganism dies out, to I₀. The controller 30 includes a memory. A table where each specific microorganism is related to a corresponding dose with which no less than 99% of the related microorganism dies out, is recorded in the memory. The controller 30 searches the table for the selected specific microorganism, reads out a value of the dose related to this microorganism, and assigns the value to I₀. If a general sterilization process is selected by the operator, a dose enough for microorganisms commonly found in the environment to die out is assigned to I₀. Examples of I₀ in this case include values determined within the range of 50.0 mJ/cm² or more, preferably 50 to 100 mJ/cm². When a microorganism adhering to the work is roughly predictable depending on a type of the work, the predetermined value I₀ of the integral irradiance enough for this microorganism to die out may be fixed in advance according to types of works, to assign the fixed value to I₀ according to selection of a type of the work.

In step S13, the controller 30 prompts an operator to input time (irradiation time) t for which deep ultraviolet rays are irradiated to the work, via the input/output device 60. If the irradiation time t is input in step S13, the process moves to step S14. As described later, if the predetermined value I₀ of the integral irradiance is determined, the shortest irradiation time t′ such that the integral irradiance I reaches the predetermined value I₀ given that ultraviolet ray is irradiated at full power or power determined by a safety factor taken into consideration in the ultraviolet light sterilizer can be calculated from the maximum emission power P_{MAX} that the deep ultraviolet light source 20 can continuously show, or a value that is a product of P_{MAX} and a preset safety factor (for example, 90%, preferably 80%), and the minimum value of the adjustable distance d. Thus, the input of t may be omitted, t′ may be displayed to be known by an operator, and the input of confirming that the irradiation time is t′ may be performed. At this time, the distance d is not necessarily the minimum value in adjustable values. For example, the distance d may be an initial setting value (which is usually the optimum value for opening the door and setting the work) in view of avoiding a mechanical adjustment as far as possible.

In step S14, the controller 30 calculates emission power P of the deep ultraviolet light source 20 (unit: mW) necessary for the integral irradiance I to reach I₀ during the input irradiation time t. The integral irradiance I of deep ultraviolet rays (unit: mJ/cm²) irradiated to the work 1 during the irradiation time t is the integral of the irradiance of deep ultraviolet rays per unit area (unit: mW/cm²) at the position that is the distance d away from the deep ultraviolet light source 20, by the irradiation time t. Information on dependency of the irradiation of ultraviolet rays per unit area, on distances from the deep ultraviolet light source 20 is recorded in the memory provided for the controller 30. The attenuation rate is calculated based on the present distance d of the work 1 and the deep ultraviolet light source 20, to determine necessary emission power P. If the calculated emission power P is beyond the maximum emission power P_{MAX} that the deep ultraviolet light source 20 can continuously show, or is beyond the value that is the product of P_{MAX} and the preset safety factor (for example, 90%, more preferably 80%), a shorter distance d within adjustable values is assumed, and the emission power P is tried to be calculated again. When the emission power P equal to or less than the maximum emission power P_{MAX} is calculated, or it is determined that the emission power P is still beyond the maximum emission power P_{MAX} of the deep ultraviolet light source 20 or beyond the value that is the product of P_{MAX} and the preset safety factor (for example, 90%, preferably 80%) for the shortest distance d within the adjustable values, the process moves to step S15.

In step S15, it is determined whether a solution of feasible emission power P and a feasible distance d has been calculated or not. If an affirmative judgment is made in step S15, the process moves to step S16. If a negative judgment is made in step S15, the controller 30 outputs information for an operator via the input/output device 60 that sufficient sterilization cannot be performed during the irradiation time t of the last input, and that the bare minimum of the irradiation time is t′. Then, the process returns to step S13, and an operator is prompted to input longer irradiation time t or confirm that the irradiation time is t′.

In step S16, the emission power P, and/or the distance d between the work 1 and the deep ultraviolet light source 20 is/are adjusted. At this time, the irradiance of deep ultraviolet rays at the position the distance d away from the deep ultraviolet light source 20 depends on various factors such as the distance d, the emission intensity and the directional characteristics of the deep ultraviolet light emitting diodes 22, and the way of arranging (the manner of aligning) the deep ultraviolet light emitting diodes 22. Therefore, when the above adjustment is carried out, the relationship between the emission power P and the irradiance on the face to be irradiated (unit: mW/cm²) for each distance d is found out in advance for every real device (or a device of the same specification), and thereafter the adjustment is carried out based on this relationship.

When the emission power P and/or the distance d is/are adjusted in step S16, the priority is to carry out continuous emission or pulse emission at power P that is 90%, preferably 80% of the maximum emission power P_{MAX} without adjusting the distance d, and it is preferable that P be adjusted so that the integral irradiance I reaches I₀ within the input irradiation time t as the upper limit (rather than making P higher to shorten t). The emission power P of the deep ultraviolet light source can be controlled by controlling forward current of the deep ultraviolet light emitting diodes. The way to control of the forward current is not restricted. When a battery of approximately 1.5 to 15 V in voltage is used as a power source such as in a portable ultraviolet light sterilizer, preferably the control is carried out by an emission control circuit using a boost DC-DC converter or a charge pump that can let a forward current of 10 mA to 1000 mA, preferably 50 mA to 500 mA flow. Whereby, sufficient sterilization effect can be obtained for short irradiation time (for example, within 10 minutes, preferably within 1 minute) even if a comparatively low-voltage battery is used as a power source, although the performance of the used deep ultraviolet light emitting diodes affects the effect. To the above described emission control circuit, for example, a technique described in JP 2005-93566A, JP 2007-97251A or JP 2010-4691A is applicable.

When the distance d has to be adjusted in addition to the adjustment of the power P, the distance d is adjusted to the value calculated in step S14. That is, the actuators 24 are driven so that an actual value of the distance d equals to the value calculated in step S14, to adjust the distance d. After the power P and/or the distance d is/are adjusted as described above, the process moves to step S17.

In step S17, deep ultraviolet rays are irradiated from the deep ultraviolet light source 20 at the emission power P, which is already calculated in step S14, during the irradiation time t. Information on dependency of the emission power P of the deep ultraviolet light source 20 on the forward current and device temperature of the deep ultraviolet light emitting diodes 22, 22, . . . is recorded in the memory of the controller 30. The controller 30 controls (finely adjusts) the forward current flowing in the deep ultraviolet light emitting diodes 22, 22, . . . based on the information on the device temperature of the deep ultraviolet light emitting diodes 22, 22, . . . detected by the temperature sensor 23, and thereby, controls the emission power P of the deep ultraviolet light source 20 so as to keep the emission power at the value already calculated in step S14. At the same time, while deep ultraviolet rays are irradiated from the deep ultraviolet light source 20, the controller 30 operates the driver 50 to rotate the support 40 so as to level the dose of deep ultraviolet rays irradiated to the work 1. When the predetermined irradiation time t has passed, the controller 30 ends the supply of the electric current to the ultraviolet light emitting diodes 22, 22, . . . , and the rotation of the support 40 by the driver 50, and the process moves to step 18.

In step S18, the controller 30 outputs information for an operator via the input/output device that the sterilization process is ended. An operator may open the door 11, and take the work 1, which is completely sterilized, out of the housing 10.

Examples of the controller 30 include any embodiment having a memory where necessary information and flow for the above process are recorded, and a processing unit such as a microprocessor.

In the above description concerning the present invention, the ultraviolet light sterilizer 100 rotating the support 40 for leveling the dose of deep ultraviolet rays has been explained as an example. However, the present invention is not restricted to this embodiment. In one embodiment, the ultraviolet light sterilizer may include a driver for rotating the deep ultraviolet light source relative to the support for leveling the dose of deep ultraviolet rays to the work.

In the above description concerning the present invention, the ultraviolet light sterilizer 100 including the input/output device 60, that the predetermined value I₀ is determined based on a microorganism to die out, or a value enough for general sterilization is set in I₀ in step S12, has been explained as an example. However, the present invention is not restricted to this embodiment. In one embodiment, the ultraviolet light sterilizer may set a value enough for general sterilization in I₀ as an initial value, which makes the input of I₀ unnecessary. In one embodiment, the ultraviolet light sterilizer may set, in I₀, only a value corresponding to a microorganism to die out which is designated by an operator.

In the above description concerning the present invention, the ultraviolet light sterilizer 100 including the temperature sensor 23 which measures device temperature of the deep ultraviolet light emitting diodes 22, 22, . . . , that the controller 30 controls the emission power P of the deep ultraviolet light source 20 based on the device temperature of the deep ultraviolet light emitting diodes 22, 22, . . . , which is measured by the temperature sensor 23, has been explained as an example. However, the present invention is not restricted to this embodiment. One embodiment may be the ultraviolet light sterilizer not including the temperature sensor, wherein the emission power is not controlled based on the device temperature.

In the above description concerning the present invention, the ultraviolet light sterilizer 100 and the method for ultraviolet light sterilization each having the embodiment that the distance d between the deep ultraviolet light source 20 and the work 1 and/or the emission power P of the deep ultraviolet light source is/are controlled, so that the integral irradiance I in the predetermined irradiation time t reaches the predetermined value I₀ have been explained as examples. However, the present invention is not restricted to this embodiment. In one embodiment, as described already concerning step S13, the ultraviolet light sterilizer and the method for ultraviolet light sterilization may control the irradiation time t, so that the integral irradiance I in the irradiation time t reaches the predetermined value I₀. That is, in the above example, the priority is to perform irradiation only during the preset irradiation time t (sec). However, the priority may be to shorten actual irradiation time, and according to this priority, irradiation may be performed at a power as high as possible and the distance d may be also adjusted, so that the integral irradiance I reaches I₀ in the shortest time, and the irradiation may be ended when I reaches I₀. In this case, preferably, an operator is informed of the end of the irradiation by sound etc.

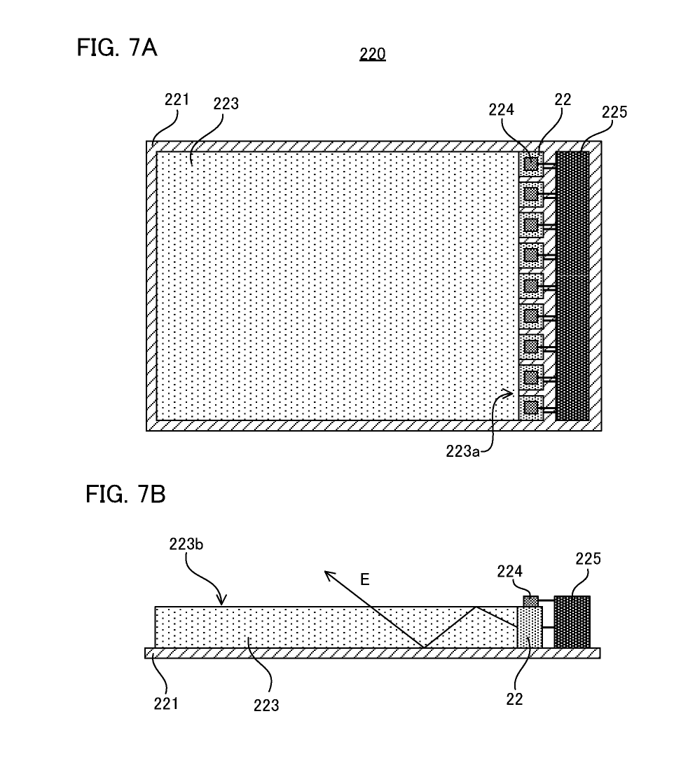

In the above description concerning the present invention, the ultraviolet light sterilizer 100 including the deep ultraviolet light source 20, which is shown in FIG. 3 as the deep ultraviolet light source, has been explained as an example. However, the present invention is not restricted to this embodiment. In one embodiment, the ultraviolet light sterilizer may include a deep ultraviolet light source 220 shown in FIG. 7, which is described later, as the deep ultraviolet light source. At least one deep ultraviolet light source 20 may be placed on any face(s) other than the top face 10c, that is, any face(s) of the front face 10a, the back face 10b, and the side faces 10e and 10f. Further, the support 40 may be composed of an ultraviolet transmitting material, and the deep ultraviolet light source 20 may be arranged on the bottom face 10d.

In the above description concerning the present invention, the embodiment of rotating the support 40 for leveling the dose of deep ultraviolet rays irradiated to the work 1 is employed. However, the means for leveling the dose is not restricted to this embodiment. The support may reciprocate by sliding, or the deep ultraviolet light source 20 may move without movement of the work.

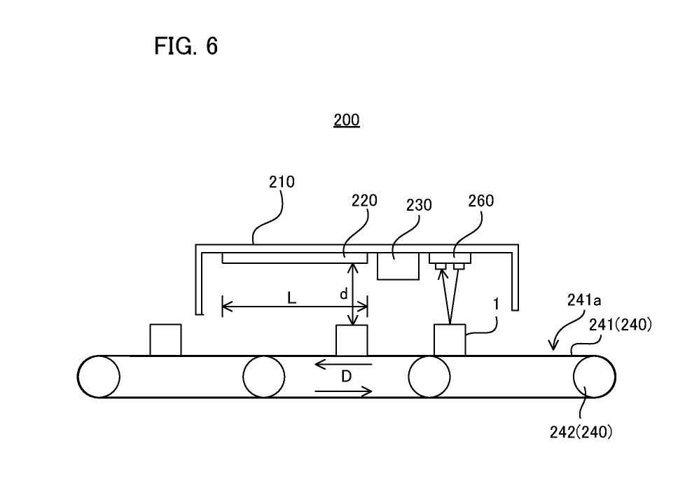

FIG. 6 is an explanatory schematic cross-sectional view of an ultraviolet light sterilizer 200 according to another embodiment of the first aspect of the present invention. In FIGS. 6-8, the same reference numerals as in FIGS. 1-5 are added to the elements which are also shown in FIGS. 1-5, and description thereof is omitted. The ultraviolet light sterilizer 200 has a conveyor 240 that moves with the work 1 placed thereon; the deep ultraviolet surface light source 220 according to one embodiment of the second aspect of the present invention, which is arranged above the conveyor 240, opposite to the conveyor 240; a controller 230; a distance sensor 260 that is arranged on the upstream side of the deep ultraviolet surface light source 220 along the conveyor 240; and a housing 210 that holds the ultraviolet surface light source 220, the distance sensor 260 and the controller 230 at predetermined positions.

The conveyor 240 has a belt 241 having no end, and having a supporting surface 241a for placing the work 1 thereon; and a driver 242 that drives the belt 241 in the direction of the arrows D in FIG. 6. The driver 242 is connected to the controller 230.

FIG. 7A is a plan view of the deep ultraviolet surface light source 220, and FIG. 7B is a side view of the deep ultraviolet surface light source 220. The deep ultraviolet light source 220 has a substrate 221; a light guide plate 223 that is arranged on the substrate 221; a plurality of the deep ultraviolet light emitting diodes 22, 22, . . . arranged on the substrate 221, so as to be opposite to an end 223a of the light guide plate 223; temperature sensors 224, 224, . . . that measure device temperature of the deep ultraviolet light emitting diodes 22 (for example, a thermoelectric thermometer etc. Hereinafter they may be simply referred to as “temperature sensor 224”); and a current controller 225 that controls forward current of the deep ultraviolet light emitting diodes. Deep ultraviolet rays emitted from the deep ultraviolet light emitting diodes 22 goes into the light guide plate 223 from the end 223a, propagate inside the light guide plate 223 while totally reflected as the arrow E in FIG. 7B, and exit from a light emitting surface 223b of the light guide plate 223. The deep ultraviolet surface light source 220 is arranged so that the light emitting surface 223b of the light guide plate 223 is opposite to the supporting surface 241a of the conveyor 240.

For example, a plurality of the deep ultraviolet light emitting diodes 22 can be used as “an ultraviolet light-emitting module comprising: a cylindrical or polygonal columnar base body; a plurality of ultraviolet light-emitting devices disposed on a side surface of the base body such that a light axis of each ultraviolet light-emitting device passes through a center axis of the base body to emit ultraviolet rays radially relative to the center axis; and a cover formed of an ultraviolet transmitting material, the cover covering the base body and being air-tightly mounted to the base body such that inside thereof is filled with an inert gas or dried air; and a flow path for a cooling medium formed inside the base body accommodated for flowing a cooling medium through the flow path” as disclosed in JP 5591305 B. Such a module makes it possible to increase the intensity of ultraviolet rays, and keep the device temperature fixed.

The current controller 225 is connected to the temperature sensor 224, the deep ultraviolet light emitting diodes 22, and the controller 230. The current controller 225 receives information on device temperature of the deep ultraviolet light emitting diodes 22 from the temperature sensor 224. The current controller 225 also receives, from the controller 230, information on the distance d between the light emitting surface 223b of the light guide plate 223 of the deep ultraviolet surface light source 220 and the work 1, and information on the irradiance of deep ultraviolet rays (unit: mW/cm²) to be achieved at the position the distance d away from the light emitting surface 223b. The irradiance of deep ultraviolet rays at the position the distance d away from the light emitting surface 223b depends on the distance d, the emission intensity of the deep ultraviolet light emitting diodes 22, and the characteristics of the light guide plate. The emission intensity of the deep ultraviolet light emitting diodes 22 depends on the forward current of the deep ultraviolet light emitting diodes 22, and the device temperature of the deep ultraviolet light emitting diodes 22. The current controller 225 includes a memory and a processing unit. The relationship between the irradiance of deep ultraviolet rays at the position the distance d away from the light emitting surface 223b, and the distance d and the emission intensity of the deep ultraviolet light emitting diodes 22 is recorded as the first function; and the relationship between the emission intensity, and the forward current and the device temperature of the deep ultraviolet light emitting diodes 22 is recorded as the second function in the memory of the current controller 225. The current controller 225 calculates a necessary emission intensity of the deep ultraviolet light emitting diodes 22 using the first function, from the information on the distance d between the light emitting surface 223b and the work 1, and the information on the irradiance of deep ultraviolet rays to be achieved at the position the distance d away from the light emitting surface 223b, which are received from the controller 230. Then the forward current to flow in the deep ultraviolet light emitting diodes 22 are calculated using the second function, from the calculated necessary emission intensity of the deep ultraviolet light emitting diodes 22, and the information on the device temperature of the deep ultraviolet light emitting diodes 22, which is received from the temperature sensor 224, to let the deep ultraviolet light emitting diodes 22 emit light by the calculated forward current. While the deep ultraviolet light emitting diodes 22 emit light, the current controller 225 continues to monitor the information on the device temperature received from the temperature sensor 224, and controls the forward current using the second function so as to keep the emission intensity of the deep ultraviolet light emitting diodes 22 at the previously calculated necessary emission intensity.

FIG. 6 is referred to again. The distance sensor 260 is arranged above the conveyor 240 and opposite to the supporting surface 241a of the conveyor 240, and is arranged on the upstream side of the deep ultraviolet surface light source 220 along the conveyor 240. The distance sensor 260 detects the work 1, and also measures the distance to the work 1. The distance sensor 260 measures the distance to the work 1 placed on the supporting surface 241a of the conveyor 240, and transfers information on the measurement results to the controller 230. For example, a known distance sensor such as a ultrasonic distance sensor, an infrared distance sensor, or a laser distance sensor may be employed as the distance sensor 260 without particular limitation. The controller 230 converts the distance information received from the distance sensor 260 into the distance d between the light emitting surface 223b of the light guide plate 223 of the deep ultraviolet surface light source 220 and the work 1 when the work 1 comes beneath the light guide plate 223 of the deep ultraviolet surface light source 220.

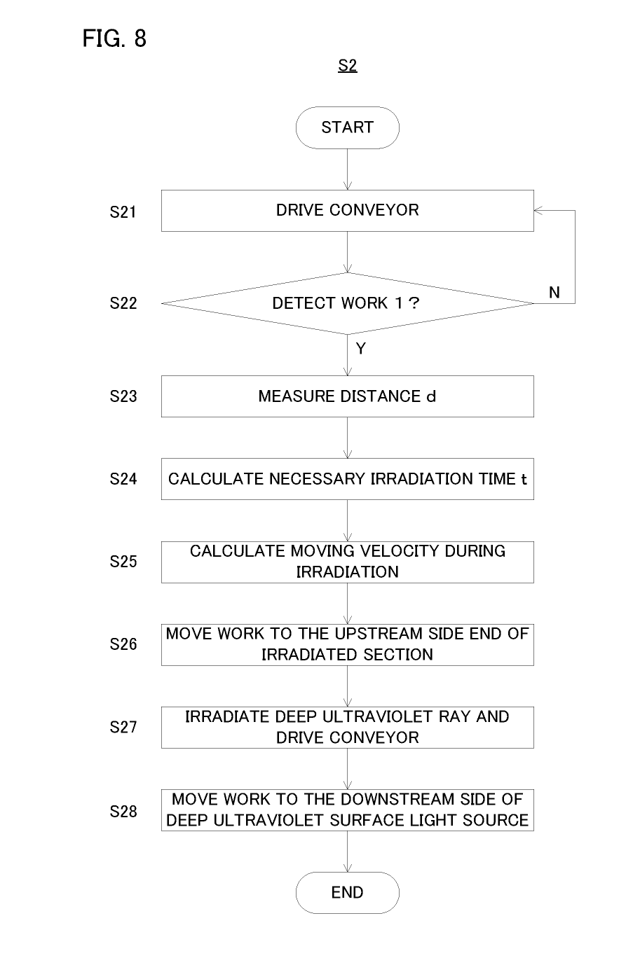

The controller 230 is connected to the current controller 225 of the deep ultraviolet surface light source 220, the distance sensor 260, and the driver 242 of the conveyor 240, to control their operations. FIG. 8 is an explanatory flowchart of a control process S2 of the ultraviolet light sterilizer 200. FIG. 8 is also an explanatory flowchart of the method for ultraviolet light sterilization according to another embodiment of the present invention at the same time. The control process S2 includes steps S21 to S28. Operation of the controller 230 will be described with reference to FIG. 8.

In step S21, the conveyor 240 is started to be driven, and the process moves to step S22. In step S22, it is determined whether the work 1 has been transferred such that it is under the distance sensor 260 or not. This determination can be made by detecting change occurring in the distance information which is received by the controller 230 from the distance sensor 260 arranged opposed to the supporting surface 241a of the conveyor 240. If an affirmative judgment is made in step S22, the process moves to next step S23. If a negative judgment is made in step S22, the process returns to step S21.

In step S23, the distance sensor 260 measures the distance to the work 1, and transfers the measurement result to the controller 230. The controller 230 receives the information on the measurement result from the distance sensor 260, and calculate the distance d between the deep ultraviolet surface light source 220 and the work 1 (that is, the distance between the light emitting surface 223b of the light guide plate 223 and the work 1) when the work 1 comes into an irradiation area beneath the deep ultraviolet surface light source 220, based on the information.

In step S24, the controller 230 calculates the irradiation time t of deep ultraviolet rays which is necessary for the work 1 to be sufficiently sterilized, based on the value of the integral irradiance I₀ of deep ultraviolet rays to be irradiated to the work 1 (unit: mJ/cm²), and the value of the distance d calculated in step S23. The ultraviolet light sterilizer 200 is configured so as to perform a sterilization process in which microorganisms commonly found in the environment sufficiently die out, and employs, for example, the value of 50.0 mJ/cm² or more as I₀. The controller 230 includes at least a memory and an processing unit. The value of I₀, and the value of the emission power P (unit: mW) to be exhibited by the deep ultraviolet surface light source 220 are recorded in the memory. The irradiance of deep ultraviolet rays (unit: mW/cm²) at the position the distance d away from the deep ultraviolet surface light source 220 depends on the emission power P of the deep ultraviolet surface light source 220 (unit: mW), and the distance d. The relationship between the irradiance of deep ultraviolet rays at the position the distance d away from the deep ultraviolet surface light source 220, and the distance d when the deep ultraviolet surface light source 220 is driven at the predetermined emission power P, the power P being recorded in the memory, is recorded in the memory of the controller 230 as a function. The controller 230 calculates the irradiance of deep ultraviolet rays over the surface of the work 1 using the function, from the previously calculated distance d, and calculates the time t necessary to achieve the integral irradiance I₀, from the calculated irradiance.

In step S25, the controller 230 calculates the velocity at which the work 1 is to be moved during the irradiation of deep ultraviolet rays to the work 1, that is, the moving velocity of the conveyor 240, from the necessary time t calculated in step S24. Length L of the section on the conveyor 240, where deep ultraviolet rays of a fixed intensity is irradiated from the deep ultraviolet surface light source 220 (see FIG. 6), is recorded in the memory of the controller 230. The moving velocity v of the conveyor 240 while deep ultraviolet rays are irradiated to the work 1 is calculated by the formula v=L/t.

In step S26, the controller 230 drives the conveyor 240, to move the work 1 to the upstream side end of the section where deep ultraviolet rays of a fixed intensity are irradiated from the deep ultraviolet surface light source 220 (see FIG. 6).

In step S27, the controller 230 transfers the value of the distance d, and the value of the irradiance to be achieved at the position the distance d away from the deep ultraviolet surface light source 220, to the current controller 225 of the deep ultraviolet surface light source 220, and transmits a command for starting the irradiation of deep ultraviolet rays. At the same time, the controller 230 controls the driver 242 of the conveyor 240 so that the moving velocity v of the conveyor 240 becomes the value calculated in step S25. After the irradiation of deep ultraviolet rays for the above described predetermined time t is finished, the process moves to step S28.

In step S28, the controller 230 further operates the conveyor 240, to move the work 1 to the downstream side of the deep ultraviolet surface light source 220.

Examples of the controller 230 include any embodiment having a memory where necessary information and flow for the above process are recorded, and a processing unit such as a microprocessor.

In the above description concerning the present invention, the ultraviolet light sterilizer 200 and the method for ultraviolet light sterilization that control the irradiation time t by the moving velocity of the conveyor 240 to achieve the predetermined integral irradiance I₀ while the emission power P of the deep ultraviolet surface light source 220 is fixed, have been explained as examples. However, the present invention is not restricted to this embodiment. In one embodiment, the ultraviolet light sterilizer and the method for ultraviolet light sterilization may control the emission power P of the deep ultraviolet light source so that the predetermined integral irradiance I₀ is achieved while the work is passing through under the deep ultraviolet light source (that is, an area opposite to the deep ultraviolet light source), while the moving velocity of the conveyor (that is, the irradiation time t) is fixed. In one embodiment, the ultraviolet light sterilizer and the method for ultraviolet light sterilization may, while the moving velocity of the conveyor (that is, the irradiation time t) is fixed, (1) calculate the emission power P of the deep ultraviolet light source at which the predetermined integral irradiance I₀ is achieved while the work passes through the area under the deep ultraviolet light source, and (2a) achieves the predetermined integral irradiance I₀ only by controlling the emission power P without any change in the moving velocity of the conveyor if the calculated value of the emission power P is no more than the feasible maximum value, or (2b) achieves the predetermined integral irradiance I₀ at a feasible emission power P by reducing the moving velocity of the conveyor, to make the irradiation time t longer, if the calculated value of the emission power P is more than the feasible maximum value.

In the above description concerning the present invention, the ultraviolet light sterilizer 200 wherein the controller 230 indirectly controls the deep ultraviolet light emitting diodes 22 via the current controller 225 of the ultraviolet surface light source 220 has been explained as an example. However, the present invention is not restricted to this embodiment. One embodiment may be the ultraviolet light sterilizer wherein the deep ultraviolet light emitting diodes 22 and the temperature sensor 224 are connected to the controller 230, and the controller 230 directly reads out the measurement value of the temperature sensor 224 and drives the deep ultraviolet light emitting diodes 22.

In the above description concerning the present invention, the deep ultraviolet surface light source 220 including the temperature sensor 224 that measures the device temperature of the deep ultraviolet light emitting diodes 22 has been explained as an example. However, the deep ultraviolet surface light source of the present invention is not restricted to this embodiment. In one embodiment, the deep ultraviolet surface light source may include a temperature adjusting device that controls the device temperature of the deep ultraviolet light emitting diodes 22 (for example, a Peltier device) instead of, or along with the temperature sensor 224.

### REFERENCE SIGNS LIST

1 work

100, 200 ultraviolet light sterilizer

10, 210 housing

10a front face

10b back face

10c top face

10d bottom face

10e, 10f side face

11 door

20 deep ultraviolet light source

21, 221 substrate

22 deep ultraviolet light emitting diodes

23, 224 temperature sensor

24 actuator

30, 230 controller

40 support

50 driver

60 input/output device

220 deep ultraviolet surface light source

223 light guide plate

223a end (of the light guide plate)

223b light emitting surface (of the light guide plate)

225 current controller

240 conveyor

241 belt

241a supporting surface (of the conveyor)

242 driver

260 distance sensor

1-21. (canceled)

22. An ultraviolet light sterilizer comprising:
a supporting surface on which a work is placed;
a deep ultraviolet light source comprising one or more deep ultraviolet light emitting diode and irradiating a deep ultraviolet ray toward the work placed on the supporting surface; and
a controller determining a combination of (1) an irradiation time t (unit: second) defined as a time for which the deep ultraviolet ray is irradiated to the work, (2) a distance d (unit: cm) between the work and the deep ultraviolet light source, and (3) an emission power P (unit: mW) of the deep ultraviolet light source, such that an integral irradiance I (unit: mJ/cm²) of the deep ultraviolet ray irradiated to the work during the irradiation time t reaches a predetermined value I₀; adjusting the irradiation time t, the distance d, or the emission power P, or combination thereof; and thereafter making the deep ultraviolet light source irradiate the deep ultraviolet ray toward the work.

23. A method for ultraviolet light sterilization of a work placed on a supporting surface by means of a deep ultraviolet light source,
the deep ultraviolet light source comprising one or more deep ultraviolet light emitting diode and emitting a deep ultraviolet ray toward the work,
the method comprising the steps of:
(a) determining a combination of (1) an irradiation time t (unit: second) defined as a time for which the deep ultraviolet ray is irradiated to the work, (2) a distance d (unit: cm) between the work and the deep ultraviolet light source, and (3) an emission power P (unit: mW) of the deep ultraviolet light source, such that an integral irradiance I (unit: mJ/cm²) of the deep ultraviolet ray irradiated to the work during the irradiation time t reaches a predetermined value I₀; and
(b) adjusting the irradiation time t, the distance d, or the emission power P, or combination thereof. based on a result determined in the step (a), and thereafter irradiating the deep ultraviolet ray to the work from the deep ultraviolet light source.

24. The ultraviolet light sterilizer according to claim 22, further comprising:
an actuator adjusting the distance d.

25. The ultraviolet light sterilizer according to claim 22,
wherein the controller calculates the emission power of the deep ultraviolet light source necessary for the integral irradiance I to reach the predetermined value I₀, given that the irradiation time t is an irradiation time inputted by an operator, and that an initial value of the distance d is a distance between the deep ultraviolet light source and the work at the time when the work is placed on the supporting surface; and
the controller determines the combination of the irradiation time t, the distance d, and the emission power P, based on the calculated emission power.

26. The ultraviolet light sterilizer according to claim 25,
wherein the controller makes a decision on whether or not the calculated emission power is greater than a maximum emission power of the deep ultraviolet light source or an emission power which is a product of the maximum emission power and a predetermined safety factor;
when the decision is negative, the controller sets the emission power P to the calculated emission power; and
when the decision is affirmative, the controller determines the combination of the irradiation time t, the distance d, and the emission power P based on the calculated emission power, by:
retrying to calculate the emission power necessary for the integral irradiance I to reach the predetermined value Io on an assumption that the distance d has a value less than the initial value; or
setting the irradiation time t at a new value greater than the irradiation time inputted by the operator, and retrying to calculate the emission power necessary for the integral irradiance I to reach the predetermined value I₀; or
setting the emission power P of the deep ultraviolet light source at the maximum emission power or at the emission power which is the product of the maximum emission power and the safety factor, and setting the irradiation time t at a shortest irradiation time t′ such that the integral irradiance I reaches the predetermined value I₀ given that the ultraviolet ray is irradiated at the emission power P.

27. The method for ultraviolet light sterilization according to claim 23, the method further comprising the steps of:
(c) placing the work on the supporting surface; and
(d) determining the integral irradiance I₀ (unit: mJ/cm²) of the deep ultraviolet ray to be irradiated to the work,
the step (a) comprising the steps of:
(a1) setting the irradiation time t; and
(a2) calculating the emission power of the deep ultraviolet light source necessary for the integral irradiance I to reach the predetermined value I₀ given that the deep ultraviolet ray is irradiated for the irradiation time t and that an initial value of the distance d is a distance between the deep ultraviolet light source and the work at the time when the work is placed on the supporting surface; and determining the combination of the irradiation time t, the distance d and the emission power P by setting the “(3) emission power P” at the calculated emission power, setting the “(1) irradiation time t” at the irradiation time set in the step (a1), and setting the “(2) distance d” at the initial value of the distance.

28. The method for ultraviolet light sterilization according to claim 27,
the step (a) further comprising the steps of:
(a3) deciding whether or not the emission power calculated in the step (a2) is greater than a maximum emission power of the deep ultraviolet light source or an emission power which is a product of the maximum emission power and a predetermined safety factor; and
(a4) when a negative decision is made in the step (a3), determining the combination of the irradiation time t, the distance d and the emission power P by setting the “(3) emission power P” of the deep ultraviolet light source at the emission power calculated in the step (a2), setting the “(1) irradiation time t” at the irradiation time set in the step (a1), and setting the “(2) distance d” at the initial value in the step (a2); and when an affirmative decision is made in the step (a3),
retrying to calculate the emission power necessary for the integral irradiance I to reach the predetermined value I₀ on an assumption that the distance d has a value less than the initial value, and determining the combination of the irradiation time t, the distance d and the emission power P by setting the “(3) emission power P” at the calculated emission power, setting the “(1) irradiation time t” at the irradiation time set in the step (a1), and setting the “(2) distance d” at the assumed value of the distance d less than the initial value, or
setting the irradiation time t at a new value greater than the irradiation time set in the step (a1), and retrying to calculate the emission power necessary for the integral irradiance I to reach the predetermined value I₀, and determining the combination of the irradiation time t, the distance d and the emission power P by setting the “(3) emission power P” at the calculated emission power, setting the “(1) irradiation time t” at the new value of the irradiation time, and setting the “(2) distance d” at the initial value of the distance in the step (a2), or
determining the combination of the irradiation time t, the distance d and the emission power P by setting the “(2) distance d” at the initial value of the distance in the step (a2), setting the “(3) emission power P” of the deep ultraviolet light source at the maximum emission power or at the emission power which is the product of the maximum emission power and the safety factor, and setting the “(1) irradiation time t” at a shortest irradiation time t′ such that the integral irradiance I reaches the predetermined value I₀ given that the ultraviolet ray is irradiated at the emission power P.
